(19) United States
(12) Patent Application Publication
(10) Pub. No.: US 2026/0206123 A1
(43) Pub. Date: Jul. 16, 2026

(54) SYSTEMS, APPARATUSES, AND METHODS OF MANUFACTURING LINEAR ACCELERATORS

(71) Applicant: Varian Medical Systems, Inc., Palo Alto, CA (US)

(72) Inventors: Alexander CAHILL, San Francisco, CA (US); Flavio POEHLMANN-MARTINS, Fremont, CA (US); Christopher WALZ, Sunnyvale, CA (US); Ogy SABEV, San Jose, CA (US); Joshua MCNEUR, San Jose, CA (US); Joshua MOODY, Livermore, CA (US); Raiyan SEEDE, San Mateo, CA (US)

(73) Assignee: Varian Medical Systems, Inc., Palo Alto, CA (US)

(21) Appl. No.: 19/023,737

(22) Filed: Jan. 16, 2025

(51) Int. Cl. H05H 7/18; H05H 9/04

(52) U.S. Cl. CPC H05H 7/18; H05H 9/048; H05H 2277/11

(57) ABSTRACT

A linear accelerator for a radiation therapy system includes a plurality of cavities aligned along a length of the linac and a channel extending between adjacent cavities of the plurality of cavities. The plurality of cavities include a first material and the channel includes a second material different from the first material.

### TECHNICAL FIELD

The present disclosure relates to systems, apparatuses, and methods of manufacturing linear accelerators for radiation therapy systems.

### BACKGROUND

Optimization of radiation therapy systems involves increasing accelerating gradients in cavities of linear accelerators to delay electric breakdown within the cavities.

### SUMMARY

At least one example embodiment relates to a linear accelerator (“linac”) for a radiation therapy system. The linac may include a plurality of cavities aligned along a length of the linac and a channel extending between adjacent cavities of the plurality of cavities. The plurality of cavities may include a first material and the channel may include a second material different from the first material.

In at least one example embodiment, the channel may include an insert coupled between adjacent cavities of the plurality of cavities.

In at least one example embodiment, the channel may be formed from the first material and may include a coating of the second material.

In at least one example embodiment, each cavity of the plurality of cavities may include a nose on each side of the cavity adjacent to a next cavity along the length of the linac. In at least one example embodiment, the channel may extend between noses of adjacent cavities of the plurality of cavities. In at least one example embodiment, the channel may include a tip cap abutting each nose of each cavity. In at least one example embodiment, the channel may be formed from the first material and the tip caps may be formed from the second material. In at least one example embodiment, the channel may be formed from the first material and the tip caps may be formed by coating a portion of the channel abutting each nose with the second material. In at least one example embodiment, each nose of each cavity of the plurality of cavities may be squared such that the channel extends perpendicularly from each nose.

In at least one example embodiment, the first material may be at least one of copper or a copper-silver alloy.

In at least one example embodiment, the second material may be stainless steel.

In at least one example embodiment, the second material may be at least one of titanium, vanadium, cobalt 31, or Molybdenum.

Also described herein is a method of manufacturing a linear accelerator (“linac”). The method may include forming a plurality of cavities from a first material and forming a channel extending between the noses of the plurality of cavities. Each cavity of the plurality of cavities may include a nose on each side of the cavity adjacent to a next cavity along a length of the linac. The channel may include a second material.

In at least one example embodiment, the forming the channel may include forming the channel from the first material and coating the channel with the second material. In at least one example embodiment, the coating the channel with the second material may include at least one of electroplating or sputtering the second material onto the first material of the channel. In at least one example embodiment, the coating the channel with the second material may include forming a tip cap abutting each nose with the second material. A portion of the channel of the first material may extend between a first tip cap of a first nose of a first cavity and a second tip cap of a second nose of a second cavity.

In at least one example embodiment, the forming the channel may include coupling the channel to each nose of each cavity by at least one of brazing or cryogenic shrink fitting.

In at least one example embodiment, the first material may be at least one of copper or a copper-silver alloy.

In at least one example embodiment, the second material may be stainless steel.

In at least one example embodiment, the second material may be at least one of titanium, vanadium, cobalt 31, or Molybdenum.

In at least one example embodiment, the method may further include treating at least a portion of each of the plurality of cavities with a surface treatment configured to harden the portion of each of the plurality of cavities treated with the surface treatment. In at least one example embodiment, the surface treatment may be laser peening.

### BRIEF DESCRIPTION OF THE DRAWINGS

The various features and advantages of the non-limiting embodiments herein may become more apparent upon review of the detailed description in conjunction with the accompanying drawings. The accompanying drawings are merely provided for illustrative purposes and should not be interpreted to limit the scope of the claims. The accompanying drawings are not to be considered as drawn to scale unless explicitly noted. For purposes of clarity, various dimensions of the drawings may have been exaggerated.

FIG. 1 illustrates a linear accelerator according to example embodiments.

FIG. 2 illustrates a portion of the linear accelerator of claim 1 including a channel with an insert according to example embodiments.

FIG. 3 is a graph illustrating a relationship between a radius of an insert and properties of a linear accelerator according to example embodiments.

FIG. 4 illustrates the portion of the linear accelerator of claim 2 including a channel with tip caps according to example embodiments.

FIG. 5 illustrates the portion of the linear accelerator of claim 2 including a channel and a coating along an exterior of the cavities according to example embodiments.

FIG. 6 illustrates a simulation used to determine where to apply surface treatments according to example embodiments.

FIG. 7 is a method of manufacturing a linear accelerator according to example embodiments.

### DETAILED DESCRIPTION OF EXAMPLE EMBODIMENTS

Independent of the grammatical term usage, individuals with male, female or other gender identities are included within the term.

Some detailed example embodiments are disclosed herein. However, specific structural and functional details disclosed herein are merely representative for purposes of describing some example embodiments. Example embodiments may, however, be embodied in many alternate forms and should not be construed as limited to only example embodiments set forth herein.

Accordingly, while example embodiments are capable of various modifications and alternative forms, example embodiments thereof are shown by way of example in the drawings and will herein be described in detail. It should be understood, however, that there is no intent to limit an example embodiment to the particular forms disclosed, but to the contrary, example embodiments are to cover all modifications, combinations, equivalents, and alternatives falling within the scope of an example embodiment. Like numbers refer to like elements throughout the description of the figures.

It should be understood that when an element or layer is referred to as being “on,” “connected to,” “coupled to,” or “covering” another element or layer, it may be directly on, connected to, coupled to, or covering the other element or layer or intervening elements or layers may be present. In contrast, when an element is referred to as being “directly on,” “directly connected to,” or “directly coupled to” another element or layer, there are no intervening elements or layers present. Like numbers refer to like elements throughout the specification. As used herein, the term “and/or” includes any and all combinations of one or more of the associated listed items.

It should be understood that, although the terms first, second, third, etc. may be used herein to describe various elements, regions, layers and/or sections, these elements, regions, layers, and/or sections should not be limited by these terms. These terms are only used to distinguish one element, region, layer, or section from another region, layer, or section. Thus, a first element, region, layer, or section discussed below could be termed a second element, region, layer, or section without departing from the teachings of example embodiment.

Spatially relative terms (e.g., “beneath,” “below,” “lower,” “above,” “upper,” and the like) may be used herein for ease of description to describe one element or feature's relationship to another element(s) or feature(s) as illustrated in the figures. It should be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the term “below” may encompass both an orientation of above and below. The device may be otherwise oriented (rotated 90 degrees or at other orientations) and the spatially relative descriptors used herein interpreted accordingly.

The terminology used herein is for the purpose of describing various example embodiment only and is not intended to be limiting of example embodiment. As used herein, the singular forms “a,” “an,” and “the” are intended to include the plural forms as well, unless the context clearly indicates otherwise. It will be further understood that the terms “includes,” “including,” “comprises,” and/or “comprising,” when used in this specification, specify the presence of stated features, integers, steps, operations, and/or elements, but do not preclude the presence or addition of one or more other features, integers, steps, operations, elements and/or groups thereof.

Example embodiments are described herein with reference to cross-sectional illustrations that are schematic illustrations of example embodiment. As such, variations from the shapes of the illustrations are to be expected. Thus, example embodiment should not be construed as limited to the shapes of regions illustrated herein but are to include deviations and variations in shapes.

When the words “about” and “substantially” are used in this specification in connection with a numerical value, it is intended that the associated numerical value include a tolerance of ±10% around the stated numerical value, unless otherwise explicitly defined. Moreover, when the terms “generally” or “substantially” are used in connection with geometric shapes, it is intended that precision of the geometric shape is not required but that latitude for the shape is within the scope of the disclosure. Furthermore, regardless of whether numerical values or shapes are modified as “about,” “generally,” or “substantially,” it will be understood that these values and shapes should be construed as including a manufacturing or operational tolerance (e.g., ±10%) around the stated numerical values or shapes.

Unless otherwise defined, all terms (including technical and scientific terms) used herein have the same meaning as commonly understood by one of ordinary skill in the art to which example embodiment belong. It will be further understood that terms, including those defined in commonly used dictionaries, should be interpreted as having a meaning that is consistent with their meaning in the context of the relevant art and will not be interpreted in an idealized or overly formal sense unless expressly so defined herein.

Optimization of radiation therapy systems involves increasing accelerating gradients in cavities of linear accelerators to delay electric breakdown within the cavities. One way to increase accelerating gradients is to blunt noses of the cavities. However, blunting noses of the cavities reduces radio frequency (“RF”) efficiency of the linear accelerator. To offset the reduction in radio frequency efficiency, larger radio frequency sources may be used. However, larger radio frequency sources may increase both cost and size of linear accelerators.

Example embodiments described herein increase accelerating gradients of linear accelerators while also increasing and/or maintaining RF efficiency within linear accelerators. Typically linear accelerators are made of a material with a relatively high electrical conductivity to minimize wall losses within the linear accelerator. For example, copper may be used due to its relatively high electrical conductivity. However, copper has a low breakdown threshold when compared to other metals. In contrast, stainless steel has a relatively low electrical conductivity but a high breakdown threshold. For example, copper has a direct current (“DC”) threshold voltage of about 175 mega volts per meter (MV/m) and a square root thermal conductivity of about 20 watts per meter Kelvin to the one half power ((W/mK)^{1/2}). Stainless steel has a DC threshold voltage of about 825 MV/m and a square root thermal conductivity of about 4 (W/mK)^{1/2}. Example embodiments herein describe linear accelerators that utilize different materials at different portions of the linear accelerator to provide linear accelerators with optimal accelerating gradients and RF efficiency.

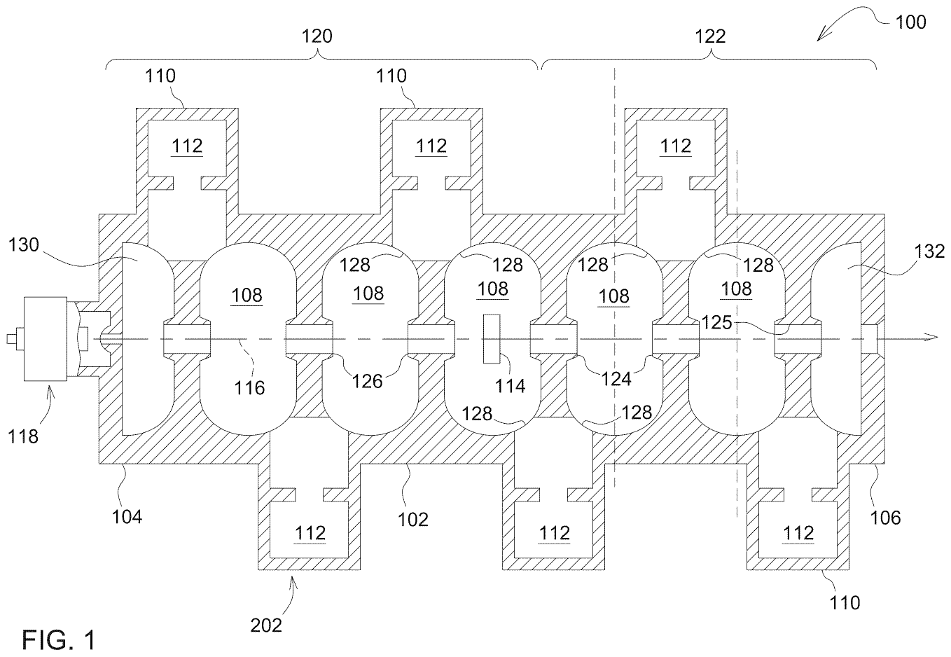

FIG. 1 is a schematic axial sectional view of an example linear accelerator (“linac”) 100. The linac 100 includes a main body 102 having a first end 104, a second end 106, and a chain of electromagnetically coupled resonant cavities 108 between the first end 104 and the second end 106. The electromagnetically coupled resonant cavities 108 may be referred to herein as electromagnetic cavities 108 and/or cavities 108. The linac 100 also includes a plurality of coupling bodies 110, each of which having a coupling cavity 112 that couples to two adjacent cavities 108. The linac 100 is excited by microwave power delivered by a microwave source at a frequency near its resonant frequency, for example, between 1000 MHz and 20 GHz, and more preferably, between 2800 and 3000 MHz. The linac 100 may be excited by power having other levels in other embodiments by an energy source. The microwave source may be a Magnetron or a Klystron. The power may enter one cavity 108, preferably one of the cavities along the chain, through an opening 114.

During use, a linear beam 116 of electrons is injected into the linac 100 by an electron gun source 118 at the first end 104. The linear beam 116 may be either continuous or pulsed. The linear beam 116 passes through a first section 120 of the linac 100 in which electrons are captured and accelerated, and enters a second section 122 of the linac 100 where the captured electrons are further accelerated.

In the illustrated embodiments, the electromagnetic cavities 108 are doughnut shaped with aligned central beam apertures 124 which permit passage of the beam 116 through a channel 125 extending between adjacent cavities 108. The cavities 108 may each have noses 126 that project from each side of a cavity 108 of optimized configuration in order to improve efficiency of interaction of the microwave power and electron beam. In at least one example embodiment, the channel 125 may extend between the noses 126 of adjacent cavities 108. The cavities 108 are electromagnetically coupled together through the coupling cavities 112, each of which is coupled to each of the adjacent pair of cavities 108 by an opening 128. The coupling cavities 112 are resonant at the same frequency as the cavities 108 and do not interact with the beam 116.

The chain includes a first cavity 130 and a last cavity 132. In at least one example embodiment, the first cavity 130 and the last cavity 132 have one-half of volume of one of the cavities 108. Alternatively, the first cavity 130 and the last cavity 132 may have the same size and shape as the cavities 108 or a volume different than one-half the volume of the cavities 108.

The spacing between cavities 108 is about one-half of a free-space wavelength, so that electrons accelerated in one cavity 108 will arrive at the next cavity 108 in a right phase relative to the microwave field for additional acceleration. Alternatively, the cavities 108 can have other spacing. In some embodiments, most of the cavities 108 and most of the coupling cavities 112 are similar such that the fields in most of the cavities 108 are substantially the same. Alternatively, the cavities 108 and/or the coupling cavities 112 can have other configurations such that the fields in some of the cavities 108 are different.

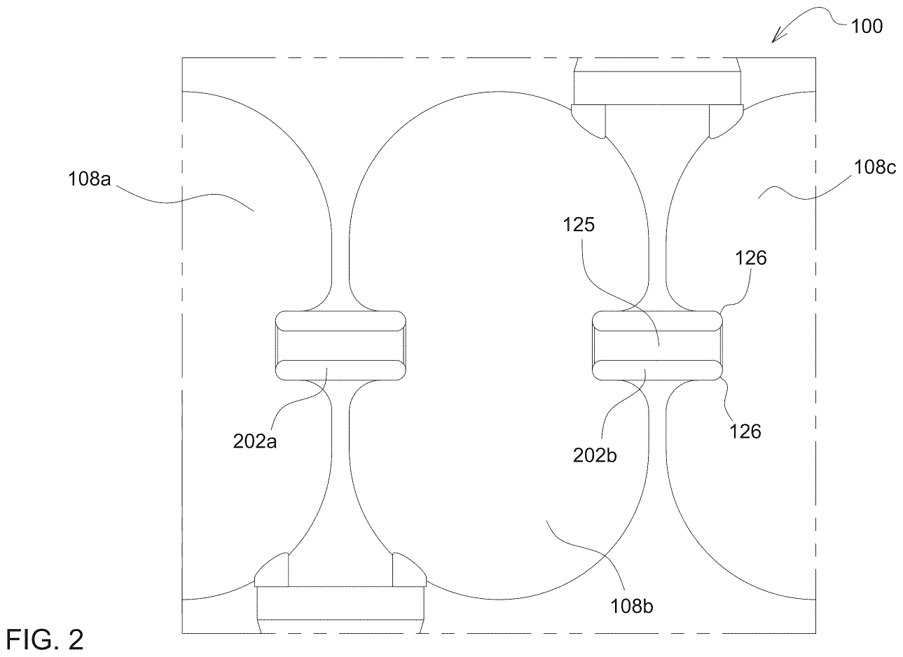

FIG. 2 illustrates a portion of the linac 100 including a portion of a first cavity 108a, a second cavity 108b, and a portion of a third cavity 108c. Thus, the first cavity 108a, the second cavity 108b, and the third cavity 108c are aligned along a length of the linac 100 and the channel 125 extends between the first cavity 108a and the second cavity 108b and between the second cavity 108b and the third cavity 108c and similarly along the length of the linac.

In at least one example embodiment, the channel 125 includes an insert coupled between adjacent cavities. In particular, a first insert 202a is coupled between the first cavity 108a and the second cavity 108b and a second insert 202b is coupled between the second cavity 108b and the third cavity 108c. An insert may be coupled between adjacent cavities 108 of the entire chain of cavities 108 along the length of the linac 100.

In at least one example embodiment, each of the inserts may be fixed to the cavities 108 via a bonding technique such as brazing or cryogenic shrink fitting although methods of attachment are not limited herein.

In at least one example embodiment, each cavity 108 of the plurality of cavities 108 of the linac 100 may be formed from a first material. Each of the inserts including the first insert 202a and the second insert 202b may be formed from a second material. The first material may be different from the second material. In at least one example embodiment, each of the inserts including the first insert 202a and the second insert 202b may be formed from the first material and may include a coating of the second material on an end of the insert coupled to the cavity 108.

In at least one example embodiment, the first material may be a material with a relatively high electrical conductivity and/or a high arcing threshold. In at least one example embodiment, the first material may be a metal or an insulator that may be coated with a metal. For example, the first material may be at least one of copper or a copper-silver alloy. Alternatively or additionally, the first material may be a coating such as diamond or graphene or another material that has both a high electrical conductivity and a high arcing threshold that may be coated on top of a base material of the linac. The example materials described herein are not limiting and a material with a relatively high electrical conductivity may be used as the first material. A material may have a relatively high electrical conductivity when the material has greater than or equal to 90% of the conductivity of copper. In particular, materials with 90% on the International Annealed Copper Standard (IACS) may be considered materials with a relatively high electrical conductivity.

In at least one example embodiment, the second material may be a material with a high breakdown threshold. A high breakdown threshold may be correlated to a hardness of a material. In particular, hardness of a material has been shown to be correlated with increased breakdown performance of the material. Thus, materials with a hardness of about 800 MPa, double the Vicker's hardness of Copper, may be considered sufficiently hard and may have a high breakdown threshold. The electrical and thermal conductivity of the second material may be less than that of the first material. For example, the second material may be at least one of stainless steel, titanium, vanadium, cobalt 31, or Molybdenum.

By forming the inserts with the second material or via the first material with a coating of the second material, the second material may not materially impact the overall electrical and thermal conductivity of the cavities 108. However, the breakdown threshold along the channel 125, the most vulnerable location of the linac 100, may be increased by including inserts of the second material. In at least one example embodiment, the channel 125 may be considered the most vulnerable location because the electric field is concentrated at this location of the linac 100. By increasing the breakdown threshold along the channel 125, the noses 126 of the cavities 108 may be made sharper or less blunted which may increase both the efficiency and the accelerating gradient of the linac. In particular, each nose 126 of each cavity 108 may be squared such that the channel 125 extends perpendicularly from each nose 126.

In at least one example embodiment, simulation methods and scaling methods may be used to examine surface fields of the linac 100. A radius of the inserts of the linac 100 may be optimized when a likelihood of damage to the channel 125 is minimized. In at least one example embodiment, a radius of the inserts of the linac 100 may be measured from a center of the insert to an outer edge of the insert. The simulation methods and scaling methods may balance pulse heating against a risk of RF breakdown to minimize a likelihood of damage to the channel 125.

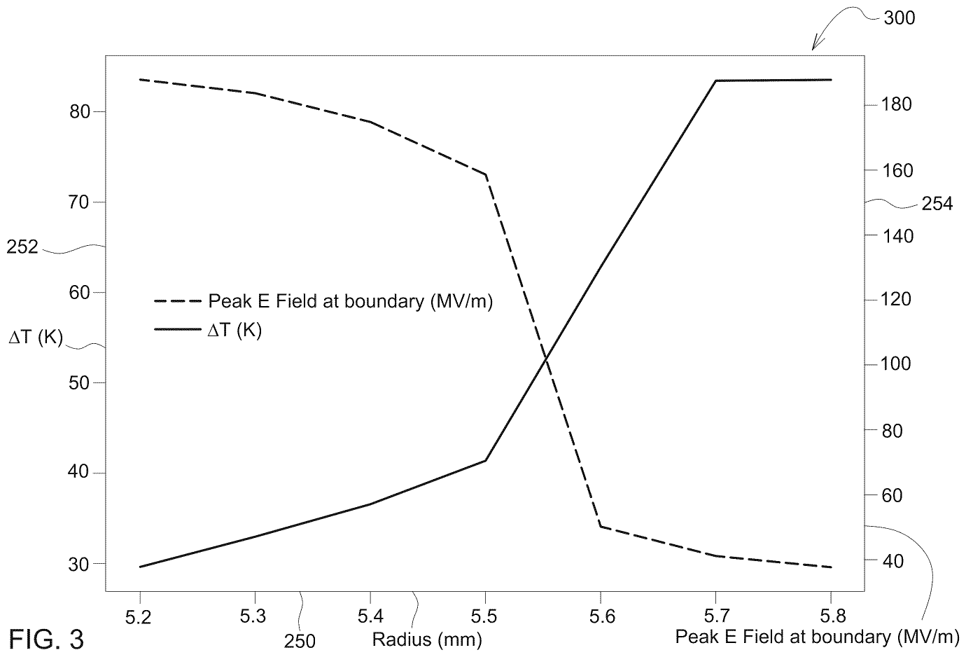

FIG. 3 is a graph 300 illustrating a relationship between a radius 250 of an insert, a pulse temperature 252 of the second material and a peak electric field 254 at a boundary between the second material and the first material of the linac 100. The first material of the graph 300 of FIG. 3 is copper and the second material is stainless steel. A radius of an insert such as the insert 202a or 202 b may be about 5.55 mm when the first material is copper and the second material is stainless steel to optimize RF breakdown versus pulse heating effects of the linac 100. Different optimizations may result in different radii for different materials. Thus, a radius of an insert for the linac 100 may be determined by optimizing RF breakdown versus pulse heating effects for specific materials of the linac 100 and the insert such as the first insert 202a and the second insert 202b.

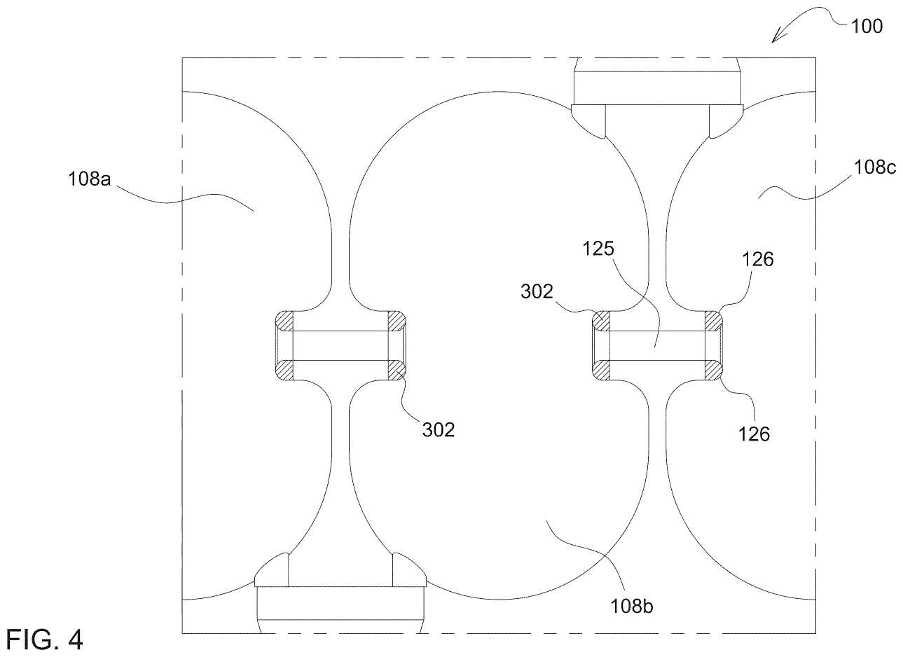

FIG. 4 illustrates the portion of the linac 100 including the portion of a first cavity 108a, the second cavity 108b, and the portion of a third cavity 108c of FIG. 1. In at least one example embodiment, the channel 125 includes tip caps 302 abutting each nose 126 of each of the cavities 108 of the entire chain of cavities 108 along the length of the linac 100. In at least one example embodiment, the tip caps 302 may be an insert coupled between the noses 126 of the cavities 108 and the channel 125. In at least one example embodiment, each of the tip caps 302 may be fixed to the cavities 108 via a bonding technique such as brazing or cryogenic shrink fitting although methods of attachment are not limited herein. Alternatively or additionally, the tip caps 302 may be a coating formed on a portion of the channel 125 that abuts the nose 126 of each of the cavities 108.

In at least one example embodiment, the cavities 108 and the channel 125 may be formed from the first material as described above with respect to FIG. 2. The tip caps 302 may be formed from the second material as described above with respect to FIG. 2.

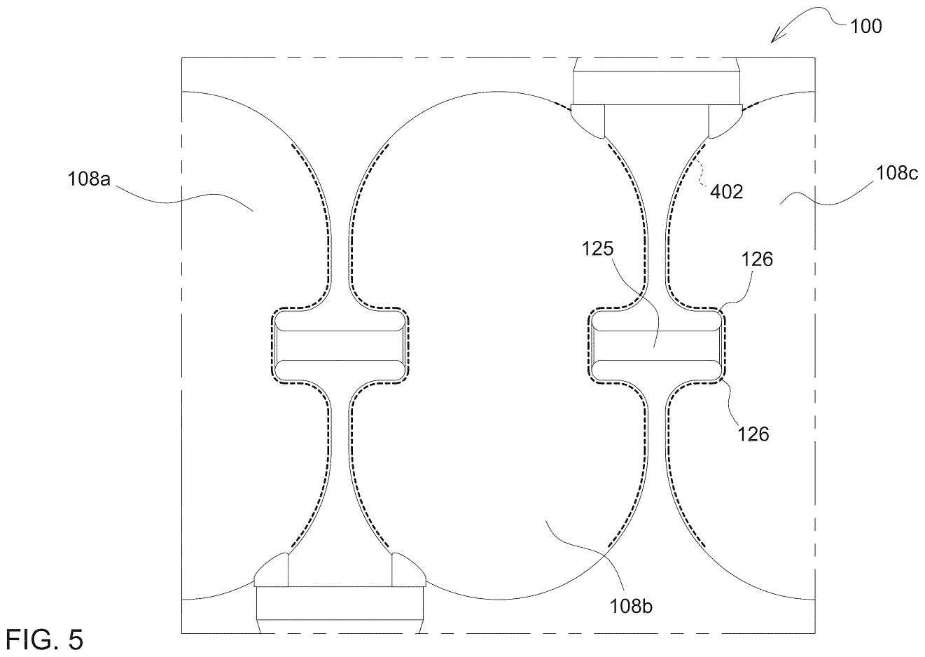

FIG. 5 illustrates the portion of the linac 100 including the portion of the first cavity 108a, the second cavity 108b, and the portion of the third cavity 108c of FIG. 1. Each of the first cavity 108a, the second cavity 108b, and the third cavity 108c includes a surface treatment configured to harden the portion of each of the first cavity 108a, the second cavity 108b, and the third cavity 108c treated with the surface treatment. In at least one example embodiment, the linac 100 may either include inserts such as the first insert 202a and the second insert 202b along a length of the channel 125 or may include surfaces treated with a surface treatment. In at least one example embodiment, the coatings described above with reference to FIG. 4 are surface treatments that may be applied to at least a portion of the first cavity 108a, the second cavity 108b, and the third cavity 108c.

In at least one example embodiment, an interior portion 402 of each of the first cavity 108a, the second cavity 108b, and the third cavity 108c may be treated with the surface treatment. In at least one example embodiment, locations to be treated with the surface treatment may be determined based on simulations. For example, simulation methods and scaling methods may be used to examine surface fields of the linac 100. Locations that are determined to be at risk of damage due to RF breakdown may be treated with a surface treatment. In at least one example embodiment, the surface treatments may be configured to harden the portion of each of the first cavity 108a, the second cavity 108b, and the third cavity 108c treated with the surface treatment to a desired harness. For example, the portion of each of the first cavity 108a, the second cavity 108 b, and the third cavity 108 c treated with the surface treatment may be hardened to at least 800 MPa, double the Vicker's hardness of Copper, such that each of the portion of each of the first cavity 108a, the second cavity 108b, and the third cavity 108c treated with the surface treatment are considered sufficiently hard and have a high breakdown threshold.

In at least one example embodiment the surface treatment may be laser peening. Alternatively or additionally, the surface treatment may be one of chemical vapor deposition or physical vapor deposition such as high energy plasma vapor deposition. In at least one example embodiment, at least one of titanium nitride or molybdenum disulfide may be used in a surface treatment of chemical vapor deposition or physical vapor deposition.

In at least one example embodiment, the cavities 108 may be formed from the first material as described above with respect to FIG. 2. The inserts, including the insert 202a and 202b, and the coating 402 may be formed from the second material as described above with respect to FIG. 2.

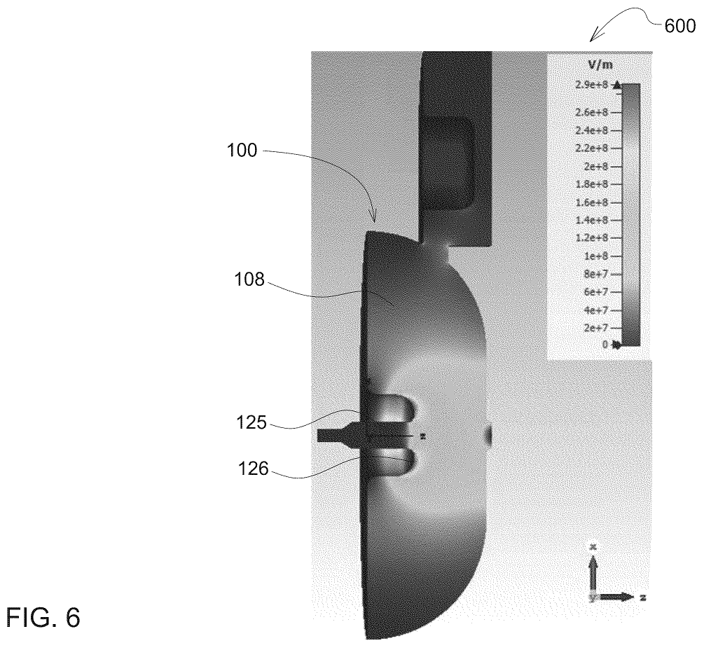

FIG. 6 illustrates an example simulation 600 used to determine where to apply surface treatments. The simulation 600 shows a portion of the linac 100. The simulation 600 shows a peak electric field of the linac 100. As shown in the simulation 600, the peak electric field may be larger at areas near the nose 126 and the channel 125 of the cavity 108 than areas of the cavity 108 further away from the nose 126 and the channel 125. The peak electric field may be similar for additional cavities 108 of the cavities along the chain of the linac 100. Thus, the surface treatments may be applied similarly to each cavity 108 of a plurality of cavities of the linac 100.

In at least one example embodiment, the surface treatment may be applied at locations with a peak electric field above a threshold. For example, a surface treatment may be applied to locations with a peak electric field above about 1.5*10⁸ V/m. In at least one example embodiment, locations with a peak electric field above the threshold may be at risk of damage due to RF breakdown. Thus, application of the surface treatment at these locations may reduce the risk of RF breakdown in the linac 100.

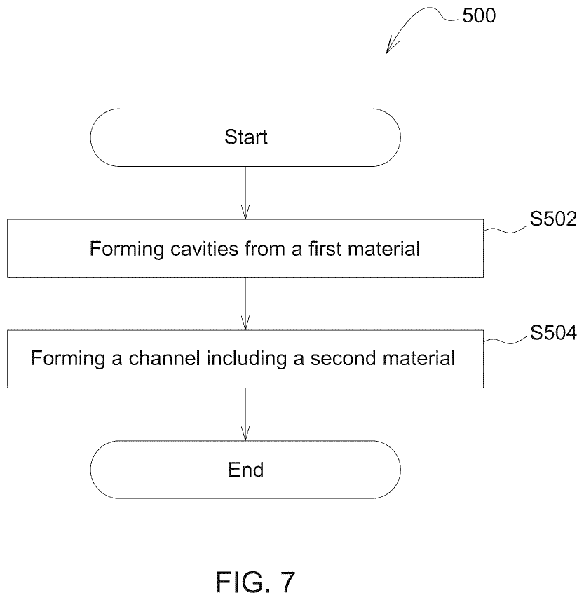

FIG. 7 is a flow chart of a method 500 of manufacturing a linac. FIG. 7 will be described with respect to the linac 100 of FIGS. 1-4. The method 500 begins at step S502 where the cavities 108 are formed from a first material such as any of the first materials described above with respect to FIG. 2. At step S504, the channel 125 is formed that includes a second material such as any of the second materials described above with respect to FIG. 2.

In at least one example embodiment, forming the channel 125 may include coupling the channel to each nose 126 of each cavity 108 of the linac 100. The channel 125 may be coupled to each nose 126 of each cavity 108 of the linac by at least one of brazing or cryogenic shrink fitting.

In at least one example embodiment, the channel 125 may be an insert such as the first insert 202a and the second insert 202b described in FIG. 2. In at least one example embodiment, forming the channel 125 with the second material includes forming the channel 125 of the first material and coating the channel 125 with the second material. Coating the channel 125 may include at least one of electroplating, sputtering, vapor deposition, chemical plasma spraying, or vacuum plasma spraying the second material onto the first material of the channel 125.

In at least one example embodiment, coating the channel 125 with the second material may include forming the tip cap 302 abutting each nose 126 with the second material. A portion of the channel 125 extending from a first tip cap of a first cavity to a second tip cap of a second cavity adjacent to the first cavity may be formed from the first material.

In at least one example embodiment, the method 500 may further include performing a surface treatment on the interior portion 402 of each of the cavities 108 of the linac. As described above, the surface treatment may be configured to harden the portion of the cavities 108 treated with the surface treatment. In at least one example embodiment the surface treatment may be laser peening. Alternatively or additionally, the surface treatment may be one of chemical vapor deposition or physical vapor deposition.

The above-described systems, apparatuses, and methods provide improved linear accelerators for use in radiation therapy systems. The linear accelerators described herein may have increased RF efficiency as well as increased accelerating gradients. Thus, costs of the linear accelerator may be reduced by not requiring a higher efficiency RF source. Additionally, a size of the linear accelerator may be reduced by not requiring the higher efficiency RF source.

Example embodiments have been disclosed herein, it should be understood that other variations may be possible. Such variations are not to be regarded as a departure from the spirit and scope of the present disclosure, and all such modifications as would be obvious to one skilled in the art are intended to be included within the scope of the following claims.

### NON-LIMITING ILLUSTRATIVE EMBODIMENTS

The following is a list of non-limiting illustrative embodiments disclosed herein:

Illustrative embodiment 1 includes a linear accelerator (“linac”) for a radiation therapy system. The linac comprises a plurality of cavities aligned along a length of the linac and a channel extending between adjacent cavities of the plurality of cavities. The plurality of cavities include a first material and the channel includes a second material different from the first material.

Illustrative embodiment 2 includes the linac of illustrative embodiment 1, wherein the channel includes an insert coupled between adjacent cavities of the plurality of cavities.

Illustrative embodiment 3 includes the linac of any one of illustrative embodiments 1 and 2, wherein the channel is formed from the first material and includes a coating of the second material.

Illustrative embodiment 4 includes the linac of any one of illustrative embodiments 1, 2, and 3, wherein each cavity of the plurality of cavities includes a nose on each side of the cavity adjacent to a next cavity along the length of the linac.

Illustrative embodiment 5 includes the linac of illustrative embodiment 4, wherein each cavity of the plurality of cavities includes a nose on each side of the cavity adjacent to a next cavity along the length of the linac.

Illustrative embodiment 6 includes the linac of any one of illustrative embodiments 4 and 5, wherein the channel includes a tip cap abutting each nose of each cavity.

Illustrative embodiment 7 includes the linac of illustrative embodiment 6, wherein the channel is formed from the first material and the tip caps are formed from the second material.

Illustrative embodiment 8 includes the linac of any one of illustrative embodiments 6 and 7, wherein the channel is formed from the first material and the tip caps are formed by coating a portion of the channel abutting each nose with the second material.

Illustrative embodiment 9 includes the linac of any one of illustrative embodiments 4, 5, 6, 7, and 8, wherein each nose of each cavity of the plurality of cavities is squared such that the channel extends perpendicularly from each nose.

Illustrative embodiment 10 includes the linac of any one of illustrative embodiments 1, 2, 3, 4, 5, 6, 7, 8, and 9, wherein the first material is at least one of copper or a copper-silver alloy.

Illustrative embodiment 11 includes the linac of any one of illustrative embodiments 1, 2, 3, 4, 5, 6, 7, 8, 9, and 10, wherein the second material is stainless steel.

Illustrative embodiment 12 includes the linac of any one of illustrative embodiments 1, 2, 3, 4, 5, 6, 7, 8, 9, 10, and 11 wherein the second material is at least one of titanium, vanadium, cobalt 31, or Molybdenum.

Illustrative embodiment 13 includes a method of manufacturing a linear accelerator (“linac”). The method comprises forming a plurality of cavities from a first material and forming a channel extending between the noses of the plurality of cavities. Each cavity of the plurality of cavities includes a nose on each side of the cavity adjacent to a next cavity along a length of the linac and the channel includes a second material.

Illustrative embodiment 14 includes the method of illustrative embodiment 13, wherein the forming the channel includes forming the channel from the first material and coating the channel with the second material.

Illustrative embodiment 15 includes the method of illustrative embodiment 14, wherein the coating the channel with the second material includes at least one of electroplating or sputtering the second material onto the first material of the channel.

Illustrative embodiment 16 includes the method of any one of illustrative embodiments 14 and 15, wherein the coating the channel with the second material includes forming a tip cap abutting each nose with the second material, and a portion of the channel of the first material extends between a first tip cap of a first nose of a first cavity and a second tip cap of a second nose of a second cavity.

Illustrative embodiment 17 includes the method of any one of illustrative embodiments 13, 14, 15, and 16, wherein the forming the channel includes coupling the channel to each nose of each cavity by at least one of brazing or cryogenic shrink fitting.

Illustrative embodiment 18 includes the method of any one of illustrative embodiments 13, 14, 15, 16, and 17, wherein the first material is at least one of copper or a copper-silver alloy.

Illustrative embodiment 19 includes the method of any one of illustrative embodiments 13, 14, 15, 16, 17, and 18, wherein the second material is stainless steel.

Illustrative embodiment 20 includes the method of any one of illustrative embodiments 13, 14, 15, 16, 17, 18, and 19, wherein the second material is at least one of titanium, vanadium, cobalt 31, or Molybdenum.

Illustrative embodiment 21 includes the method of any one of illustrative embodiments 13, 14, 15, 16, 17, 18, 19, and 20, further comprising treating at least a portion of each of the plurality of cavities with a surface treatment configured to harden the portion of each of the plurality of cavities treated with the surface treatment.

Illustrative embodiment 22 includes the method of illustrative embodiment 21, wherein the surface treatment is laser peening.

We claim:

1. A linear accelerator (“linac”) for a radiation therapy system, the linac comprising:
a plurality of cavities aligned along a length of the linac; and
a channel extending between adjacent cavities of the plurality of cavities,
wherein the plurality of cavities include a first material and the channel includes a second material different from the first material.

2. The linac of claim 1, wherein the channel includes an insert coupled between adjacent cavities of the plurality of cavities.

3. The linac of claim 1, wherein the channel is formed from the first material and includes a coating of the second material.

4. The linac of claim 1, wherein each cavity of the plurality of cavities includes a nose on each side of the cavity adjacent to a next cavity along the length of the linac.

5. The linac of claim 4, wherein the channel extends between noses of adjacent cavities of the plurality of cavities.

6. The linac of claim 4, wherein the channel includes a tip cap abutting each nose of each cavity.

7. The linac of claim 6, wherein the channel is formed from the first material and the tip caps are formed from the second material.

8. The linac of claim 6, wherein the channel is formed from the first material and the tip caps are formed by coating a portion of the channel abutting each nose with the second material.

9. The linac of claim 4, wherein each nose of each cavity of the plurality of cavities is squared such that the channel extends perpendicularly from each nose.

10. The linac of claim 1, wherein the first material is at least one of copper or a copper-silver alloy.

11. The linac of claim 1, wherein the second material is stainless steel.

12. The linac of claim 1, wherein the second material is at least one of titanium, vanadium, cobalt 31, or Molybdenum.

13. A method of manufacturing a linear accelerator (“linac”), the method comprising:
forming a plurality of cavities from a first material, each cavity of the plurality of cavities including a nose on each side of the cavity adjacent to a next cavity along a length of the linac; and
forming a channel extending between the noses of the plurality of cavities, the channel including a second material.

14. The method of claim 13, wherein the forming the channel includes forming the channel from the first material and coating the channel with the second material.

15. The method of claim 14, wherein the coating the channel with the second material includes at least one of electroplating or sputtering the second material onto the first material of the channel.

16. The method of claim 14, wherein the coating the channel with the second material includes forming a tip cap abutting each nose with the second material, and a portion of the channel of the first material extends between a first tip cap of a first nose of a first cavity and a second tip cap of a second nose of a second cavity.

17. The method of claim 13, wherein the forming the channel includes coupling the channel to each nose of each cavity by at least one of brazing or cryogenic shrink fitting.

18. The method of claim 13, wherein the first material is at least one of copper or a copper-silver alloy.

19. The method of claim 13, wherein the second material is stainless steel.

20. The method of claim 13, wherein the second material is at least one of titanium, vanadium, cobalt 31, or Molybdenum.

21. The method of claim 13, further comprising:
treating at least a portion of each of the plurality of cavities with a surface treatment configured to harden the portion of each of the plurality of cavities treated with the surface treatment.

22. The method of claim 21, wherein the surface treatment is laser peening.
